(19) United States
(12) Patent Application Publication
(10) Pub. No.: US 2011/0039313 A1
(43) Pub. Date: Feb. 17, 2011

(54) METHOD FOR THE FERMENTATIVE PRODUCTION OF CADAVERINE

(76) Inventors: Stefan Verseck, Hilden (DE); Harald Haeger, Luedinghausen (DE); Andreas Karau, Vieux Moulin (FR); Lothar Eggeling, Juelich (DE); Hermann Sahm, Juelich (DE)

Correspondence Address: OBLON, SPIVAK, MCCLELLAND MAIER & NEUSTADT, L.L.P., 1940 DUKE STREET, ALEXANDRIA, VA, 22314, US

(21) Appl. No.: 12/517,923

(22) PCT Filed: Jan. 10, 2008
(86) PCT No.: PCT/EP2008/050222

§ 371 Date: Jun. 5, 2009

(30) Foreign Application Priority Data
Feb. 1, 2007 (DE) 102007005072.2

(51) Int. Cl. C12P 13/00; C12N 1/21; C12N 1/11; C12N 15/63

(52) U.S. Cl. 435/128; 435/252.3; 435/252.31; 435/252.32; 435/252.33; 435/252.35; 435/258.2; 435/320.1

(57) ABSTRACT

The invention relates to recombinant microorganisms in which polynucleotides which code for lysine decarboxylase are enhanced and, using which, cadaverine (1,5-diaminopentane) is produced fermentatively, with the carbon source used preferably being renewable raw materials such as, for example, glucose, sucrose, molasses and the like.

The invention relates to recombinant microorganisms in which polynucleotides which code for lysine decarboxylase are enhanced, and, using which, cadaverine (1,5-diaminopentane) is produced fermentatively, with renewable raw materials such as, for example, glucose, sucrose, molasses and the like, preferably being used as the carbon source.

### PRIOR ART

Polyamides (PAs) are an important group of polymers from which a series of specialist plastics for the automotive, sports and lifestyle industries are obtained. Diamines are important monomeric units of these polyamides. Together with dicarboxylic acids, they condense to give a very wide range of polymers, with the chain lengths of the diamines and dicarboxylic acids determining the plastics' properties.

To date, diamines are produced chemically from petrol-based raw materials (Albrecht, Klaus et al.; Plastics; Winnacker-Kuechler: Chemische Technik (5th edition) (2005), 5 465-819) via the dicarboxylic acid intermediate, or by chemical decarboxylation of amino acids (Suyama, Kaneo. The Decarboxylation of Amino Acids (4), Yakugaku Zasshi, (1965), Vol. 85(6), 513-533).

In view of increasing oil prices, a rapid switch to the synthesis of diamines from renewable raw materials by means of biotechnological methods such as, for example, fermentation, is desirable.

In the context of this problem it has now been found that, starting from a lysine-producing microorganism, a cadaverine producer can be generated by introducing an optionally heterologous gene which codes for a lysine decarboxylase.

Organisms which are capable of producing cadaverine have already been described (Tabor, Herbert; Hafner, Edmund W.; Tabor, Celia White. Construction of an Escherichia coli strain unable to synthesize putrescine, spermidine, or cadaverine: characterization of two genes controlling lysine decarboxylase. Journal of Bacteriology (1980), 144(3), 952-6, Takatsuka Yumiko; Kamio Yoshiyuki Molecular dissection of the Selenomonas ruminantium cell envelope and lysine decarboxylase involved in the biosynthesis of a polyamine covalently linked to the cell wall peptidoglycan layer. Bioscience, biotechnology, and biochemistry (2004), 68(1), 1-19). In the attempts to increase the synthesis of cadaverine, Escherichia coli strains are used which harbour a plasmid for over-expressing the homologous lysine decarboxylase (cadA). This E. coli strain produces increased amounts of cadaverine following the overexpression of the homologous cadA gene (JP 2002-223770). In further developments, and after the culture and expression of cadA in E. coli, these organisms were employed as whole-cell catalysts for converting externally fed lysine (JP 2002-223771, JP 2004-000114, EP 1482055), it also being possible for the decarboxylase to be presented on the cell surface of E. coli (JP 2004-208646). A further method is the conversion of lysine-HCl into cadaverine by means of the isolated cadA enzyme (JP 2005-060447).

The switch from the above-described biocatalytic processes towards a fermentative process in which the product is obtained directly is a decisive improvement in both the economy and the ecology of the production process.

### OBJECT OF THE INVENTION

The inventors have made it their object to provide novel methods for the fermentative production of cadaverine from renewable raw materials.

### DESCRIPTION OF THE INVENTION

The invention relates to cadaverine-producing recombinant microorganisms with a high L-lysine titre, in which polynucleotides which code for lysine decarboxylase are present in an enhanced dose in comparison to microorganisms, which act as the parent strain, which are not modified with regard to this enzyme.

The qualifier “with a high lysine titre” indicates that the parent strains preferably take the form of L-lysine producers, which differ from the original strains such as, for example, wild-type strains in that they produce L-lysine in larger quantities and accumulate it in the cell or in the surrounding fermentation medium. The titre is measured in mass/volume (g/l).

In wild-type strains, strict regulatory mechanisms prevent the production of metabolites such as L-amino acids beyond what is needed for the cell's own consumption, and their release into the medium. The construction of strains called amino acid producers by the manufacturer therefore requires that these metabolic regulations be overcome.

Methods of mutagenesis, selection and choice of mutants are employed in order to eliminate control mechanisms and to improve the performance properties of these microorganism. In this manner, one obtains strains which are resistant to antimetabolites such as, for example, to the lysine analogue S-(2-aminoethyl)cysteine or the valine analogue 2-thiazoloalanine and which produce chemical compounds, for example L-amino acids such as L-lysine or L-valine.

For some years, methods of the recombinant DNA technology have also been employed for the targeted strain improvement of L-amino-acid-producing strains, for example of Corynebacterium glutamicum and Escherichia coli, by enhancing or diminishing individual amino acid biosynthesis genes and studying the effect on the production of the chemical compound.

Reviews on the biology, genetics and biotechnology of Corynebacterium glutamicum can be found in “Handbook of Corynebacterium glutamicum” (Eds.: L. Eggeling and M. Bott, CRC Press, Taylor & Francis, 2005), in the special edition of the Journal of Biotechnology (Chief Editor: A. Pühler) with the title “A New Era in Corynebacterium glutamicum Biotechnology” (Journal of Biotechnology 104/1-3, (2003)) and in the book by T. Scheper (Managing Editor) “Microbial Production of L-Amino Acids” (Advances in Biochemical Engineering/Biotechnology 79, Springer Verlag, Berlin, Germany, 2003).

The nucleotide sequence of the genome of Corynebacterium glutamicum is described in Ikeda and Nakagawa (Applied Microbiology and Biotechnology 62, 99-109 (2003)), in EP 1 108 790 and in Kalinowski et al. (Journal of Biotechnology 104/1-3, (2003)).

Suitable polynucleotides which code for lysine decarboxylase may be obtained from strains of, for example, Escherichia coli, Bacillus halodurans, Bacillus cereus, Bacillus subtilis, Bacillus thuringensis, Burkholderia ambifaria, Burkholderia vietnamensia, Burkholderia cenocepatia, Chromobacterium violaceum, Selenomonas ruminantium, Vibrio cholerae, Vibrio parahaemolyticus, Streptomyces coelicolor, Streptomyces pilosus, Eikenalla corrodens, Eubacterium acidaminophilum, Francisella tulariensis, Geobacillus kaustophilus, Salmonella typhi, Salmonella typhimurium, Hafnia alvei, Neisseria meningitidis, Thermoplasma acidophilum, Plasmodium falciparum, Kineococcus radiotolerans, Oceanobacillus iheyensis, Pyrococcus abyssi, Porochlorococcus marinus, Proteus vulgaris, Rhodoferax ferrireducens, Saccharophagus degradans, Streptococcus pneumoniae, Synechococcus sp.

Suitable lysine decarboxylases which can be employed in the process according to the invention are understood to be enzymes and their alleles or mutants which are capable of decarboxylating lysine.

The polynucleotides which are employed in accordance with the invention and which code for the enzyme lysine decarboxylase are preferably derived from Escherichia coli SEQ ID NO: 1. The latter is available free in internationally accessible databases such as, for example, that of the National Library of Medicine and the National Institute of Health (NIH) of the United States of America under the accession number NC 007946. The same sequence is also available free at the Institut Pasteur (France) on the colibri web server under the number b4131 or the gene name cadA. The same sequence is also available free through the web server ExPasy, which is maintained by the Swiss Institute of Bioinformatics, under the number P0A9H4 or the gene name cadA.

The measure of employing inventive microorganisms which produce larger amounts of L-lysine cannot be deduced from the prior art.

On the contrary, U.S. Pat. No. 5,827,698 describes that diminishing the lysine decarboxylase activity improves the L-lysine production in E. coli.

The production of cadaverine is aided by additionally overexpressing, in the cadaverine-producing recombinant cell, a polynucleotide which codes for a protein referred to as cadaverine/lysine antiporter, preferably obtained from Escherichia coli (SEQ ID NO: 3; TC 2.A.3.2.2), which facilitates the transport of the abovementioned compound from the cell into the medium. Further suitable cadaverine/lysine antiporters are derived from strains of, for example, Escherichia coli, Thermoplasma acidophilum or Vibrio cholerae.

It is also possible to use transporters which naturally export cadaverine or related diamines, or which, following mutation, attain this ability of exporting cadaverine or related diamines.

The invention also includes the overexpression of endogenous transporter genes of C. glutamicum which code for proteins which catalyze the export of cadaverine. Equally, the invention comprises that preferably no competing lysine or arginine export takes place in cadaverine-producing strains, i.e. that the corresponding export genes or export functions are present at a diminished level or are silenced.

The invention relates to recombinant microorganisms, in particular to coryneform bacteria, which contain enhanced quantities of the polynucleotides which code for the abovementioned proteins. It is preferred to enhance, in particular to overexpress, the nucleotide sequences which code for lysine decarboxylase and/or the lysine/cadaverine antiporter.

Preferred microorganisms belong to the families Enterobacteriaceae, in particular the genus Escherichia, Bacillus and in particular the species E. coli and B. subtilis, it being possible for the lysine decarboxylase which enhances the production of cadaverine to be of endogenous or exogenous origin.

The overexpressed polynucleotides which, in the recombinant microorganisms according to the invention, code for lysine decarboxylase and/or the lysine/cadaverine antiporter can originate from microorganisms of different families or genera.

Due to the overexpression of the abovementioned genes, individually or together, these microorganisms produce cadaverine to an increased extent in comparison with microorganisms in which these genes are not overexpressed.

The recombinant microorganisms according to the invention are made up by the methods of recombinant genetic engineering which are known to the skilled worker.

In general, the vectors which harbour the above-mentioned genes are introduced into the cells by conventional transformation or transfection techniques. Suitable methods can be found in Sambrook et al. (Molecular Cloning: A Laboratory Manual, 2nd ed., Cold Spring Harbor Laboratory, 1989).

The invention also relates to vectors, in particular to plasmids, which contain the polynucleotides employed in accordance with the invention and which, if appropriate, replicate in the bacteria. Equally, the invention relates to the recombinant microorganisms which have been transformed with the abovementioned vectors.

In this context, the two polynucleotides may be under the control of a single promoter, or of two promoters.

In the present context, the term “enhancement” describes the increase in the intracellular activity or concentration of one or more enzymes or proteins in a microorganism which are encoded by the DNA in question, for example by increasing the copy number of the gene(s), of the ORF(s) by at least one (1) copy, by functionally linking a strong promoter with the gene, or by using a gene or allele or ORF which codes for a suitable enzyme or protein with a high activity and, if appropriate, by combining these measures. In E. coli, lac, tac and trp are mentioned as strong promoters.

An open reading frame (ORF) designates a segment of a nucleotide sequence which codes, or can code, for a protein, or polypeptide, or ribonucleic acid to which protein/polypeptide or ribonucleic acid no function can be assigned in the state of the art. After a function has been assigned to the relevant segment of the nucleotide sequence, one generally talks about a gene. Alleles are generally understood as meaning alternative forms of a given gene. The forms are distinguished by differences in the nucleotide sequence.

Gene product generally refers to the protein encoded by a nucleotide sequence, i.e. an ORF, a gene or an allele, or the encoded ribonucleic acid.

Methods of enhancement, in particular overexpression, generally increase the activity or concentration of the protein in question by at least 10%, 25%, 50%, 75%, 100%, 150%, 200%, 300%, 400% or 500%, up to a maximum of 1000% or 2000%, based on the activity or concentration of the wild-type protein, or the activity or concentration of the protein in the microorganism or parent strain which is not recombinant for the enzyme or protein in question. A nonrecombinant microorganism or parent strain is understood as meaning the microorganism on which the enhancement or over-expression according to the invention is carried out.

The genes or gene constructs may either be present in plasmids with different copy numbers or else be integrated and amplified in the chromosome. Alternatively, an overexpression of the genes in question may furthermore be achieved by altering the media composition and the process control.

Suitable agents for increasing the copy number of the cadA alleles are plasmids which are replicated in coryneform bacteria. A large number of known plasmid vectors such as, for example, pZ1 (Menkel et al., Applied and Environmental Microbiology (1989) 64: 549-554), pEKEx1 (Eikmanns et al., Gene 102: 93-98 (1991)) or pHS2-1 (Sonnen et al., Gene 107: 69-74 (1991)) are based on the cryptic plasmids pHM1519, pBL1 or pGA1. Other plasmid vectors such as, for example, those which are based on pCG4 (U.S. Pat. No. 4,489,160), or pNG2 (Serwold-Davis et al., FEMS Microbiology Letters 66, 119-124 (1990)) or pAG1 (U.S. Pat. No. 5,158,891) may be used in the same manner. A summary of plasmid vectors of Corynebacterium glutamicum is found in Tauch et al. (Journal of Biotechnology 104 (1-3), 27-40 (2003).

The method of chromosomal gene amplification as described for example by Reinscheid et al. (Applied and Environmental Microbiology 60, 126-132 (1994)) for the duplication or amplification of the hom-thrB operon may furthermore be employed for increasing the copy number. In this method, the complete gene, or allele, is cloned into a plasmid vector which is capable of replication in a host (typically E. coli), but not in C. glutamicum. Suitable vectors are, for example, pSUP301 (Simon et al., Bio/Technology 1, 784-791 (1983)), pK18mob or pK19mob (Schäfer et al., Gene 145, 69-73 (1994)), pGEM-T (Promega Corporation, Madison, Wis., USA), pCR2.1-TOPO (Shuman, Journal of Biological Chemistry 269: 32678-84 (1994); U.S. Pat. No. 5,487,993), pCR®Blunt (Invitrogen, Groningen, the Netherlands; Bernard et al., Journal of Molecular Biology, 234: 534-541 (1993)), pEM1 (Schrumpf et al., Journal of Bacteriology 173: 4510-4516 (1991)) or pBGS8 (Spratt et al., Gene 41: 337-342 (1986)). The plasmid vector which contains the gene, or allele, to be amplified is subsequently transferred into the desired C. glutamicum strain by conjugation or transformation. The conjugation method is described for example in Schafer et al. (Applied and Environmental Microbiology 60, 756-759 (1994)). Transformation methods are described for example in Thierbach et al. (Applied Microbiology and Biotechnology 29, 356-362 (1988)), Dunican and Shivnan (Bio/Technology 7, 1067-1070 (1989)) and Tauch et al. (FEMS Microbiological Letters 123, 343-347 (1994)). Following homologous recombination by means of a cross-over event, the resulting strain contains at least two copies of the gene, or allele, in question. In particular, it is also possible to use the tandem amplification method as described in WO 03/014330 or the method of amplification by integration at a desired site as described in WO 03/040373 for increasing the copy number by at least 1, 2 or 3.

The term “diminishment” or “to diminish” describes the reduction or switching-off of the intracellular activity of one or more enzymes or proteins in a microorganism which are encoded by the corresponding DNA, for example by using a weak promoter or by using a gene, or allele, which codes for a corresponding enzyme with a low activity, or by inactivating the relevant gene or enzyme, or protein, and, if appropriate, combining these measures.

As the result of the diminishment measures, the activity or concentration of the relevant protein is generally reduced to 0 to 75%, 0 to 50%, 0 to 25%, 0 to 10% or 0 to 5% of the activity or concentration of the wild-type protein, or of the activity or concentration of the protein in the starting microorganism. A “starting microorganism” is understood as meaning the microorganism in which the diminishment of the relevant gene is carried out.

Organisms which are claimed in particular are coryneform bacteria in which the abovementioned polynucleotides which code for the enzyme lysine decarboxylase are present in an enhanced dose, preferably an overexpressed dose.

Since coryneform bacteria do not naturally contain any polynucleotide which codes for this enzyme, even the presence of one copy of a gene which codes for lysine decarboxylase and which originates from a heterologous organism is referred to as overexpression.

The invention also relates to a method of producing cadaverine in which microorganisms, in particular coryneform bacteria, are transformed with one of the abovementioned polynucleotides, the resulting recombinant bacteria are fermented in a suitable medium under conditions which are suitable for the expression of the lysine decarboxylase which is encoded by this polynucleotide, and the cadaverine formed is accumulated and isolated, if appropriate also together with further dissolved components of the fermentation liquor and/or the biomass (≧0 to 100%).

In particular, the invention relates to a method of producing cadaverine, in which the following steps are generally carried out:
a) fermentation, under conditions which are suitable for the production of the enzyme and of cadaverine, of recombinant microorganisms, in particular coryneform bacteria, in which nucleotide sequences which code for lysine decarboxylase, and preferably polynucleotides which code for a protein referred to as lysine/cadaverine antiporter, are present in an enhanced dose, in particular in an overexpressed dose, and
b) accumulation of the cadaverine in the fermentation liquor and/or in the cells of the abovementioned bacteria.

This may be followed by the isolation of the cadaverine from the fermentation liquor and/or from the cells of the abovementioned bacteria, with, if appropriate, components of the fermentation liquor and/or the biomass also being removed in part or fully, or else fully remaining in the product.

The nucleotide sequence of the cadA gene from E. coli is shown in SEQ ID NO: 1.

In the genus Corynebacterium, it is in particular the species Corynebacterium glutamicum, which is known in expert circles, that is to be mentioned. The starting materials for the microorganisms according to the invention are, for example, known wild-type strains of the species Corynebacterium glutamicum such as, for example,
Corynebacterium glutamicum ATCC13032
Corynebacterium acetoglutamicum ATCC15806
Corynebacterium acetoacidophilum ATCC13870
Corynebacterium melassecola ATCC17965
Corynebacterium thermoaminogenes FERM BP-1539
Brevibacterium flavum ATCC14067
Brevibacterium lactofermentum ATCC13869 and
Brevibacterium divaricatum ATCC14020.

Suitable precursors of the strains employed in accordance with the invention are known strains of coryneform bacteria which have the ability for producing L-lysine, such as, for example, the strains:
Corynebacterium glutamicum DM58-1/pDM6 (=DSM4697) described in EP 0 358 940,
Corynebacterium glutamicum MH20 (=DSM5714) described in EP 0 435 132,
Corynebacterium glutamicum AHP-3 (=FermBP-7382) described in EP 1 108 790, and
Corynebacterium thermoaminogenes AJ12521 (=FERM BP-3304) described in U.S. Pat. No. 5,250,423.
Corynebacterium glutamicum DM1800 (Georgi T, Rittmann D, Wendisch V F (2005) Metabolic Engineering 7: 291-301)

Strains with the name “ATCC” can be obtained from the American Type Culture Collection (Manassas, Va., USA). Strains with the name “DSM” can be obtained from the Deutsche Sammlung von Mikroorganismen and Zellkulturen (DSMZ, Braunschweig, Germany). Strains with the name “FERM” can be obtained from the National Institute of Advanced Industrial Science and Technology (AIST Tsukuba Central 6, 1-1-1 Higashi, Tsukuba Ibaraki, Japan).

Information on the taxonomical classification of strains of this group of bacteria is found, inter alia, in Kämpfer and Kroppenstedt (Canadian Journal of Microbiology 42, 989-1005 (1996)) and in U.S. Pat. No. 5,250,434. For some years (Liebl et al., International Journal of Systematic Bacteriology 41(2), 255-260 (1991)), coryneform bacteria with the species name “Brevibacterium flavum”, “Brevibacterium lactofermentum” and “Brevibacterium divaricatum” are assigned to the species Corynebacterium glutamicum. Coryneform bacteria with the species name “Corynebacterium melassecola” also belong to the species Corynebacterium glutamicum.

The microorganisms which are suitable for the measures according to the invention preferably have the ability of producing L-lysine, of accumulating it in the cell or of excreting it into the surrounding nutrient medium and accumulating it therein. In particular, the strains employed have the ability of producing >(at least) 1 g/l, ≧15 g/l, ≧20 g/l or ≧30 g/l L-lysine in ≦(a maximum of) 120 hours, ≦96 hours, ≦48 hours, ≦36 hours, ≦24 hours or ≦12 hours, before they have been transformed with the lysine decarboxylase gene. They may be strains which have been generated by mutagenesis and selection, by recombinant DNA techniques or by a combination of the two methods.

Traditional in-vivo mutagenesis methods in which mutagenic substances such as, for example, N-methyl-N′-nitro-N-nitrosoguanidine or ultraviolet light are employed may be used for the mutagenesis.

Furthermore, it is possible to use, for the muta-genesis, in-vitro methods such as, for example, a treatment with hydroxylamine (Miller, J. H.: A Short Course in Bacterial Genetics. A Laboratory Manual and Handbook for Escherichia coli and Related Bacteria, Cold Spring Harbor Laboratory Press, Cold Spring Harbor, 1992) or mutagenic oligonucleotides (T. A. Brown: Gentechnologie für Einsteiger, Spektrum Akademischer Verlag, Heidelberg, 1993) or the polymerase chain reaction (PCR) as described in the manual of Newton and Graham (PCR, Spektrum Akademischer Verlag, Heidelberg, 1994).

Further instructions for the generation of mutations can be found in the prior art and in known textbooks of genetics and molecular biology such as, for example, the textbook of Knippers (“Molekulare Genetik” [Molecular genetics], 6th edition, Georg Thieme Verlag, Stuttgart, Germany, 1995), that of Winnacker (“Gene and Klone” [Genes and clones], VCH Verlagsgesellschaft, Weinheim, Germany, 1990) or that of Hagemann (“Allgemeine Genetik” [General genetics], Gustav Fischer Verlag, Stuttgart, 1986).

When using in-vitro methods, the cadA gene, which is described in the prior art, is amplified from isolated total DNA of a wild-type strain with the aid of the polymerase chain reaction, if appropriate cloned into suitable plasmid vectors, and the DNA is then subjected to the mutagenesis method. Instructions on the amplification of DNA sequences with the aid of the polymerase chain reaction (PCR) can be found by the skilled worker in the manual of Gait: Oligonucleotide Synthesis: A Practical Approach (IRL Press, Oxford, UK, 1984) and in Newton and Graham: PCR (Spektrum Akademischer Verlag, Heidelberg, Germany, 1994), inter alia. Equally, it is also possible to use in-vitro mutagenesis methods as are described for example in the known manual by Sambrook et al. (Molecular Cloning, A Laboratory Manual, 2nd ed., Cold Spring Harbor Laboratory Press, Cold Spring Harbor, N.Y., USA, 1989). Corresponding methods are also commercially available in the form of what are known as kits, such as, for example, the “QuikChange Site-Directed Mutagenesis Kit” from Stratagene (La Jolla, USA), which has been described by Papworth et al. (Strategies 9(3), 3-4 (1996)). Suitable cadA alleles are subsequently selected and studied with the above-described methods.

The invention relates to a strain for the fermentative production of cadaverine, preferably of coryneform bacteria, in particular Corynebacterium glutamicum, which strain has at least one heterologously expressed gene which codes for a lysine decarboxylase, preferably cadA from E. coli.

Also suitable are suitable strains of the genus Escherichia.

The lysine decarboxylase allele or gene which is preferably used can be transferred into suitable strains by the gene replacement method as described by Schwarzer and Pühler (Bio/Technology 9, 84-87 (1991)) or Peters-Wendisch et al. (Microbiology 144, 915-927 (1998)). Here, the lysine decarboxylase allele in question is cloned into a vector which does not replicate in C. glutamicum, such as, for example, pK18mobsacB or pK19mobsacB (Jäger et al., Journal of Bacteriology 174: 5462-65 (1992)) or pCR®Blunt (Invitrogen, Groningen, the Netherlands; Bernard et al., Journal of Molecular Biology, 234: 534-541 (1993)), and this vector is subsequently transferred into the suitable C. glutamicum host by transformation or conjugation. Following homologous recombination by means of a first cross-over event which brings about integration and a suitable second cross-over event which brings about an excision in the target gene, or the target sequence, the mutation is successfully incorporated. Finally, it is possible to use the amplification methods described in WO 03/014330 and WO 03/040373.

In addition, it may be advantageous for the production of cadaverine simultaneously to enhance, in particular to overexpress, one or more lysine biosynthesis enzymes, in addition to the expression of the lysine decarboxylase genes or alleles employed in accordance with the invention. In general, the use of endogenous genes is preferred.

“Endogenous genes” or “endogenous nucleotide sequences” is understood as meaning the genes, or nucleotide sequences, and alleles which are present in the population of a species.

In the present context, the term “enhancement” describes the increase in the intracellular activity or concentration of one or more enzymes or proteins in a microorganism which are encoded by the DNA in question, for example by increasing the copy number of the gene(s), by using a strong promoter or by using a gene, or allele, which codes for a corresponding enzyme or protein with a high activity, and, if appropriate, combining these measures.

In addition, it may be advantageous for the improved production of cadaverine to overexpress, in the coryneform bacteria produced in the above-described manner, one or more enzymes of the respective biosynthetic pathway, of glycolysis, of anaplerosis, of the pentose phosphate cycle, of the amino acid export and, if appropriate, regulatory proteins, in order to increase the production of lysine in the claimed organisms. In general, the use of endogenous genes is preferred in the above-described measures.

Thus, it is advantageous for the increased production of L-lysine in coryneform microorganisms to overexpress one or more of the genes selected from the group consisting of:

A dapA gene which codes for a dihydrodipicolinate synthase, such as, for example, the dapA gene of the wild-type of Corynebacterium glutamicum, which gene is described in EP 0 197 335.

A zwf gene which codes for a glucose-6-phosphate dehydrogenase, such as, for example, the zwf gene of the wild-type of Corynebacterium glutamicum, which gene is described in JP-A-09224661 and EP-A-1108790.

The zwf alleles of Corynebacterium glutamicum which are described in US-2003-0175911-A1 and which code for a protein in which for example the L-alanine at position 243 of the amino acid sequence is replaced by L-threonine, or in which the L-aspartic acid at position 245 is replaced by L-serine.

The zwf alleles of Corynebacterium glutamicum which are described in WO 2005/058945 and which code for a protein in which for example the L-serine at position 8 of the amino acid sequence is replaced by L-threonine, or in which the L-glycine at position 321 is replaced by L-serine.

A pyc gene which codes for a pyruvate carboxylase, such as, for example, the pyc gene of the wild-type of Corynebacterium glutamicum, which gene is described in DE-A-198 31 609 and EP 1108790.

The pyc allele of Corynebacterium glutamicum, which allele is described in EP 1 108 790 and which codes for a protein in which L-proline at position 458 of the amino acid sequence is replaced by L-serine.

The pyc alleles of Corynebacterium glutamicum which are described in WO 02/31158 and in particular EP1325135B1, which code for proteins which incorporate one or more of the amino acid substitutions selected from the group consisting of L-valine at position 1 replaced by L-methionine, L-glutamic acid at position 153 replaced by L-aspartic acid, L-alanine at position 182 replaced by L-serine, L-alanine at position 206 replaced by L-serine, L-histidine at position 227 replaced by L-arginine, L-alanine at position 455 replaced by glycine and L-aspartic acid at position 1120 replaced by L-glutamic acid.

An lysC gene which codes for an aspartate kinase such as, for example, the lysC gene of the wild-type of Corynebacterium glutamicum, which gene is described as SEQ ID NO: 281 in EP-A-1108790 (see also accession number AX120085 and 120365), and the lysC gene described as SEQ ID NO: 25 in WO 01/00843 (see accession number AX063743).

An lysC^{FBR} allele which codes for a feedback-resistant aspartate kinase variant.

Feedback-resistant aspartate kinases are understood as meaning aspartate kinases which, in comparison with the wild form, exhibit a reduced sensitivity to inhibition by mixtures of lysine and threonine or mixtures of AEC (aminoethylcysteine) and threonine or lysine alone or AEC alone. The genes, or alleles, coding for these desensitized aspartate kinases are also referred to as lysC^{FBR} alleles. The prior art describes a large number of lysC^{FBR} alleles which code for aspartate kinase variants which incorporate amino acid substitutions in comparison with the wild-type protein. The coding region of the wild-type lysC gene of Corynebacterium glutamicum corresponds to accession number AX756575 of the NCBI database.

The following lysC^{FBR} alleles are preferred: lysC A279T (substitution of alanine at position 279 of the encoded aspartate kinase protein for threonine), lysC A279V (substitution of alanine at position 279 of the encoded aspartate kinase protein for valine), lysC S301F (substitution of serine at position 301 of the encoded aspartate kinase protein for phenylalanine), lysC T308I (substitution of threonine at position 308 of the encoded aspartate kinase protein for isoleucine), lysC S301Y (substitution of serine at position 308 of the encoded aspartate kinase protein for tyrosine), lysC G345D (substitution of glycine at position 345 of the encoded aspartate kinase protein for aspartic acid), lysC R320G (substitution of arginine at position 320 of the encoded aspartate kinase protein for glycine), lysC T311I (substitution of threonine at position 311 of the encoded aspartate kinase protein for isoleucine), lysC S381F (substitution of serine at position 381 of the encoded aspartate kinase protein for phenylalanine), lysC S317A (substitution of serine at position 317 of the encoded aspartate kinase protein for alanine) and lysC T380I (substitution of threonine at position 380 of the encoded aspartate kinase protein for isoleucine).

Especially preferred are the lysC^{FBR} allele lysC T311I (substitution of threonine at position 311 of the encoded aspartate kinase protein for isoleucine) and an lysC^{FBR} allele containing at least one substitution selected from the group consisting of A279T (substitution of alanine at position 279 of the encoded aspartate kinase protein for threonine) and S317A (substitution of serine at position 317 of the encoded aspartate kinase protein for alanine).

In contrast, an lysE gene which codes for a lysine export protein, such as, for example, the lysE gene of the wild-type Corynebacterium glutamicum, which gene is described in DE-A-195 48 222, is diminished or switched off.

A ddh gene which codes for a diaminopimelate dehydrogenase, such as, for example, the ddh gene of the wild-type Corynebacterium glutamicum, which gene is described in EP 1 108 790.

The zwa1 gene of the wild-type of Corynebacterium glutamicum, which gene codes for the Zwa1 protein (U.S. Pat. No. 6,632,644).

In the same manner, there are also claimed cadaverine-producing microorganisms of the genus Escherichia in which one or more of the E. coli genes selected from the group consisting of
a) the gene which codes for a feedback-resistant aspartate kinase, or alleles, in accordance with U.S. Pat. No. 5,827,698,
b) the gene which codes for dihydrodipicolinate synthase,
c) the gene which codes for dihydrodipicolinate reductase,
d) the gene which codes for succinyldiaminopimelate transaminase,
e) the gene which codes for succinyldiaminopimelate deacylase
   are simultaneously enhanced or overexpressed.

The microorganisms according to the invention can be grown continuously or batchwise by the batch method or the fed-batch method or the repeated-fed-batch method in order to produce cadaverine. A summary of known culture techniques is described in the textbook by Chmiel (Bioprozesstechnik 1. Einführung in die Bioverfahrenstechnik [Bioprocess technology 1. introduction to bioprocess technology] (Gustav Fischer Verlag, Stuttgart, 1991)) or in the textbook by Storhas (Bioreaktoren and periphere Einrichtungen [Bioreactors and peripheral equipment] (Vieweg Verlag, Braunschweig/Wiesbaden, 1994)).

The culture medium to be used must suitably meet the requirements of the strains in question. Descriptions of culture media for various microorganisms can be found in the manual “Manual of Methods for General Bacteriology” of the American Society for Bacteriology (Washington D.C., USA, 1981).

Carbon sources which can be used are sugars and carbohydrates such as, for example, glucose, sucrose, lactose, fructose, maltose, molasses, starch and cellulose, oils and fats such as, for example, soya oil, sunflower oil, peanut oil and coconut fat, fatty acids such as, for example, palmitic acid, stearic acid and linoleic acid, alcohols such as, for example, glycerol and ethanol, and organic acids such as, for example, acetic acid. These substances can be used individually or as a mixture.

Nitrogen sources which can be used are organic nitrogen-containing compounds such as peptones, yeast extract, meat extract, malt extract, corn steep liquor, soybean flour and urea, or inorganic compounds such as ammonium sulphate, ammonium chloride, ammonium phosphate, ammonium carbonate and ammonium nitrate. The nitrogen sources can be used individually or as a mixture.

Phosphorus sources which can be used are phosphoric acid, potassium dihydrogen phosphate or dipotassium hydrogen phosphate, or the corresponding sodium-containing salts. Moreover, the culture medium must contain salts of metals, such as, for example, magnesium sulphate or iron sulphate, which are required for growth. Finally, essential growth factors such as amino acids and vitamins may be employed in addition to the abovementioned substances. Moreover, suitable precursors may be added to the culture medium. The abovementioned materials may be added to the culture in the form of a single batch or may be fed in during the culture period in a suitable manner.

Substances which are employed for the pH control of the culture in a suitable manner are alkaline compounds such as sodium hydroxide, potassium hydroxide, ammonia or aqueous ammonia, or acidic compounds such as phosphoric acid or sulphuric acid. To control foam development, it is possible to employ antifoams such as, for example, fatty acid polyglycol esters. To maintain the stability of plasmids, it is possible to add, to the medium, suitable substances which have a selective effect, such as, for example, antibiotics. To maintain aerobic conditions, oxygen or oxygen-containing gas mixtures such as, for example, air, are introduced into the culture. The culture temperature is normally at from 20° C. to 45° C. and preferably at from 25° C. to 40° C. The culture is continued until a maximum of cadaverine has been produced, or until yield or productivity has reached a desired optimum. This aim is normally achieved within 10 hours to 160 hours.

The cadaverine produced in this manner is subsequently collected and then preferably isolated and, if appropriate, purified.

Methods for the determination of cadaverine and L-amino acids such as L-lysine are known from the prior art. The analysis can be carried out for example as described by Spackman et al. (Analytical Chemistry, 30, (1958), 1190) by anion exchange chromatography followed by ninhydrin derivatization, or else it may be effected by reversed-phase HPLC as described by Lindroth et al. (Analytical Chemistry (1979) 51: 1167-1174).

The process according to the invention is used for the improved fermentative production of cadaverine by using microorganisms with a high lysine titre in which a lysine decarboxylase gene and/or a protein referred to as lysine/cadaverine antiporter is/are overexpressed.

### EXAMPLES

### General Techniques

DNA manipulations were carried out using standard techniques as described for example in Sambrook, J. et al. (1989), Molecular Cloning: a laboratory manual, 2nd Ed., Cold Spring Harbor Laboratory Press, Cold Spring Harbor, N.Y. DNA amplifications were performed using the SAWADY Pwo-DNA polymerase (Peqlab Biotechnologie, Erlangen, Germany) or Platinum Pfx-DNA polymerase (Invitrogen, Karlsruhe, Germany). Unless otherwise specified, the polymerases were used as specified by the manufacturers. Oligonucleotides for the PCR amplifications and the introduction of restriction cleavage sites were obtained from MWG-Biotech (Ebersberg, Germany). The detection of constructed strains was performed by colony PCR using the READYMIX Taq polymerase (Sigma, Taufkirchen, Germany), and plasmid preparations. DNA fragments were purified and obtained using the MinElute Gel Extraction Kit (Quiagen, Hilden, Germany) following the manufacturer's instructions. Plasmid DNA was isolated by means of the Qiaprep spin Miniprep Kit (Quiagen, Hilden, Germany). All plasmids which were constructed were verified by restriction analysis followed by sequencing.

### Example 1

### Construction of pEKEx2cadA

pEKEx2cadA was constructed using the vector pEKEx2 (Kleinertz et al., 1991 Gene 102: 93), which permits the transcription of cloned genes under the control of the isopropyl-beta-D-thiogalactopyranoside (IPTG)-inducible tac promoter and the lac repressor system (lacIq). The 2.2 kb DNA fragment which codes for the cadA gene was amplified by means of the following oligonucleotides and DNA from Escherichia coli DH5 as template:

| SEQ ID NO 5: | |
|---|---|
| pcadAFr | 5′-ttgtcgacaaggagatatagatATGAACGTTATTGCAATATTGAATC-3′ (SalI) |
| SEQ ID NO 6: | |
| pcadARe | 5′-aaggatccTTATTTTTTGCTTTCTTCTTTCAATACC-3′ (BamHI) |

(Sequences which are complementary to the genomic sequence are printed in block capitals. Additional sites which were introduced into the amplificates were restriction cleavage sites for SalI and BamHI, and a ribosome binding site (aaggag) 8 nucleotides upstream of the start codon).

The PCR amplificate was phosphorylated with polynucleotide kinase (Roche, Basle, Switzerland) and cloned blunt-ended into the SmaI cleavage site of the vector pUC18 (Yanisch-Perron et al., 1985, Gene 33: 103-19). Identity and correctness of the insert were confirmed by sequencing. Thereafter, the 2.2 kb fragment was isolated as SalI-BamHI fragment from the pUC18 derivative and ligated with the SalI-BamHI-cut vector pEKEx2. The desired plasmids were selected by means of restriction digestion, and one of the plasmids was named pEKEx2cadA.

### Example 2

### Construction of pEKEx2cadBA

pEKEx2cadBA was constructed using the vector pEKEx2 (Kleinertz et al., 1991 Gene 102: 93), which permits the transcription of cloned genes under the control of the isopropyl-beta-D-thiogalactopyranoside (IPTG)-inducible tac promoter and the lac repressor system (lacIq). The 3.6 kb DNA fragment which codes for the cadB and the cadA gene was amplified by means of the following oligonucleotides and DNA from Escherichia coli DH5 as template:

| SEQ ID NO 7: | |
|---|---|
| pcadBAFr | 5′-ttggatccaaggagatatagatATGAGTTCTGCCAAGAAGATCG-3′ (BamHI) |
| SEQ ID NO 8: | |
| pcadBARe | 5′-aaggatccTTATTTTTTGCTTTCTTCTTTCAATACC-3′ (BamHI) |

(Sequences which are complementary to the genomic sequence are
printed in block capitals. Additional sites which were
introduced into the amplificates were restriction cleavage
sites for BamHI, and a ribosome binding site (aaggag) 8
nucleotides upstream of the start codon).

The PCR amplificate was phosphorylated with polynucleotide kinase (Roche, Basle, Switzerland) and cloned blunt-ended into the SmaI cleavage site of the vector pUC18 (Yanisch-Perron et al., 1985, Gene 33: 103-19). Identity and correctness of the insert were confirmed by sequencing. Thereafter, the 3.6 kb fragment was isolated as BamHI fragment from the pUC18 derivative and ligated with the BamHI-cut vector pEKEx2. The desired plasmids were selected by means of restriction digestion, and one of the plasmids was named pEKEx2cadBA.

### Example 3

### Obtaining Recombinant Cells

Competent cells of Corynebacterium glutamicum DM1800 (Georgi et al., Metab Eng. 7 (2005) 291-301) were prepared as described by Tauch et al. (Curr Microbiol. (2002) 45: 362-367). DNA of pEKEx2, pEKEx2cadA, and pEKEx2cadBA was introduced by means of electroporation, and transformants were selected on brain-heart agar from Merck (Darmstadt, Germany) supplemented with 50 mg/l kanamycin (FEMS Microbiol Lett., 1989, 53: 299-303). Plasmid DNA was isolated from transformants and characterized by means of a restriction digestion. This gave C. glutamicum pEKEx2, C. glutamicum pEKEx2cadA and C. glutamicum pEKEx2cadBA.

The strain C. glutamicum DM1800 is characterized by the properties (in comparison with the wild type C. glutamicum ATCC 13032): mutations in the alleles pyc P458S (pyruvate decarboxylase) and lysC T311I (aspartate kinase) which lead to an elevated lysine production (Georgi T, Rittmann D, Wendisch V F Metab Eng. 2005; 7(4): 291-301, Lysine and glutamate production by Corynebacterium glutamicum on glucose, fructose and sucrose: roles of malic enzyme and fructose-1,6-bisphosphatase. Metab Eng. 2005 July; 7(4): 291-301).

### Example 4

### Cadaverine Production Using Bacteria

The recombinant C. glutamicum DM1800 strains were grown at 30° C. overnight on complex medium CGIII (Eggeling and Bott, Eds, Handbook of Corynebacterium glutamicum., CRC Press, Taylor Francis Group) containing 25 mg/l kanamycin. Thereafter, the cells were harvested by in each case centrifugation for 5 minutes at 6000 rpm, resuspended, taken up in 0.9% NaCl, recentrifuged and finally taken up in 0.9% NaCl. This cell suspension was used to inoculate the minimal medium CGXII 4% glucose, 25 mg/l kanamycin (Eggeling and Bott, Eds, Handbook of Corynebacterium glutamicum., CRC Press, Taylor Francis Group). Thereafter, the cells were incubated at 30° C. In each case at least two independent fermentations were carried out. After 47 hours, samples were taken in order to determine cadaverine and amino acids. The determination was carried out by means of high-pressure liquid chromatography (J Chromat (1983) 266: 471-482). The result of the fermentation is shown in Table 1. Thus, the utilization of the strains which have been constructed and described constitutes a method of making possible the microbial production of cadaverine from sugar.

TABLE 1
Accumulation of cadaverine in the culture supernatant of recombinant
strains of Corynebacterium glutamicum DM1800.

| C. glutamicum DM1800 | L-lysine (mM) | Cadaverine (mM) |
|---|---|---|
| pEKEx2 | 27.9 | 0.0 |
| pEKEx2cadA | 0.1 | 33.3 |

1. A cadaverine-producing recombinant microorganism with a high lysine titre, in which polynucleotides which code for a lysine decarboxylase are present in an enhanced dose in comparison to microorganisms which are not modified with regard to this enzyme.

2. The microorganism according to claim 1, in which polynucleotides which code for a protein referred to as lysine/cadaverine antiporter are present in an enhanced dose in comparison to microorganisms which are not modified with regard to this protein.

3. The microorganism according to claim 1, in which the polynucleotides which code for lysine decarboxylase are derived from microorganisms selected from the group consisting of:
Escherichia coli, Bacillus halodurans, Bacillus cereus, Bacillus subtilis, Bacillus thuringensis, Burkholderia ambifaria, Burkholderia vietnamensia, Burkholderia cenocepatia, Chromobacterium violaceum, Selenomonas ruminantium, Vibrio cholerae, Vibrio parahaemolyticus, Streptomyces coelicolor, Streptomyces pilosus, Eikenalla corrodens, Eubacterium acidaminophilum, Francisella tulariensis, Geobacillus kaustophilus, Salmonella typhi, Salmonella typhimurium, Hafnia alvei, Neisseria meningitidis, Thermoplasma acidophilum, Plasmodium falciparum, Kineococcus radiotolerans, Oceanobacillus iheyensis, Pyrococcus abyssi, Porochlorococcus marinus, Proteus vulgaris, Rhodoferax ferrireducens, Saccharophagus degradans, Streptococcus pneumoniae, and Synechococcus sp.

4. The microorganism according to claim 1, in which the polynucleotides which code for the protein referred to as lysine/cadaverine antiporter are derived from microorganisms selected from the group consisting of: Escherichia coli, Thermoplasma acidophilum and Vibrio cholerae.

5. The microorganism according to claim 1, which takes the form of a recombinant strain of the genera Escherichia or Bacillus or coryneform bacteria.

6. The microorganism according to claim 1, in which the polynucleotides which code for the lysine decarboxylase and the protein referred to as lysine/cadaverine antiporter are derived from microorganisms of the species Escherichia coli.

7. The microorganism according to claim 1, in which overexpression takes place as the result of increasing the copy number of the abovementioned polynucleotides by at least one in comparison with the untransformed microorganism or by combination of the abovementioned polynucleotides with a stronger promoter in comparison with the original strain.

8. The microorganism according to claim 1, which takes the form of a coryneform microorganism and in which one or more of the genes from Corynebacterium selected from the group consisting of:
a) the dapA gene, which codes for dihydrodipicolinate synthase,
b) the gap gene, which codes for glyceraldehyde-3 phosphate dehydrogenase,
c) the zwf gene, which codes for glucose-6 phosphate dehydrogenase, and its alleles,
d) the pyc gene, which codes for pyruvate carboxylase, and its alleles,
e) the mqo gene, which codes for malate-quinone oxidoreductase,
f) the lysC gene, which codes for a feedback-resistant aspartate kinase, and its alleles, and
g) the zwa1 gene, which codes for the Zwa1 protein,
are simultaneously enhanced or overexpressed.

9. The microorganism according to claim 1, in which the lysE gene, which codes for a L lysine export protein, is diminished or switched off.

10. The microorganism according to claim 1, which takes the form of a microorganism of the genus Escherichia, in which one or more of the genes from E. coli selected from the group consisting of
a) the gene which codes for a feedback-resistant aspartate kinase, or alleles,
b) the gene which codes for dihydrodipicolinate synthase,
c) the gene which codes for dihydrodipicolinate reductase,
d) the gene which codes for succinyldiaminopimelate transaminase, and
e) the gene which codes for succinyldiaminopimelate deacylase
are simultaneously enhanced or overexpressed.

11. The microorganism according to claim 1, which is capable of producing L lysine before having been transformed with a lysine decarboxylase gene.

12. A vector or plasmid containing a polynucleotide which codes for a lysine decarboxylase and/or a polynucleotide which codes for a protein referred to as lysine/cadaverine antiporter.

13. The vector or plasmid according to claim 12 in which the polynucleotides are derived from Escherichia coli.

14. A method of producing cadaverine, in which
a) a microorganism according to claim 1 is fermented in a medium under conditions under which cadaverine is formed, and
b) the cadaverine is accumulated in the cells of the microorganism or in the fermentation medium.

15. The method according to claim 11, in which
a) the cadaverine is isolated, and, optionally,
b) further dissolved components of the fermentation liquor and/or the biomass in their entirety or in amounts of ≧0 to 100% remain in the product which has been isolated.

16. The method according to claim 14, in which coryneform bacteria are employed.

17. The method according to claim 14, in which microorganisms of the genus Escherichia are employed.


SEQUENCE LISTING (8 sequences)

SEQ ID NO: 1 - DNA, 2148 bp, Escherichia coli
atgaacgttattgcaatattgaatcacatgggggtttattttaaagaagaacccatccgtgaacttcatcgcgcgcttgaacgtctgaacttccagattgtttacccgaacgaccgtgacgacttattaaaactgatcgaaaacaatgcgcgtctgtgcggcgttatttttgactgggataaatataatctcgagctgtgcgaagaaattagcaaaatgaacgagaacctgccgttgtacgcgttcgctaatacgtattccactctcgatgtaagcctgaatgacctgcgtttacagattagcttctttgaatatgcgctgggtgctgctgaagatattgctaataagatcaagcagaccactgacgaatatatcaacactattctgcctccgctgactaaagcactgtttaaatatgttcgtgaaggtaaatatactttctgtactcctggtcacatgggcggtactgcattccagaaaagcccggtaggtagcctgttctatgatttctttggtccgaataccatgaaatctgatatttccatttcagtatctgaactgggttctctgctggatcacagtggtccacacaaagaagcagaacagtatatcgctcgcgtctttaacgcagaccgcagctacatggtgaccaacggtacttccactgcgaacaaaattgttggtatgtactctgctccagcaggcagcaccattctgattgaccgtaactgccacaaatcgctgacccacctgatgatgatgagcgatgttacgccaatctatttccgcccgacccgtaacgcttacggtattcttggtggtatcccacagagtgaattccagcacgctaccattgctaagcgcgtgaaagaaacaccaaacgcaacctggccggtacatgctgtaattaccaactctacctatgatggtctgctgtacaacaccgacttcatcaagaaaacactggatgtgaaatccatccactttgactccgcgtgggtgccttacaccaacttctcaccgatttacgaaggtaaatgcggtatgagcggtggccgtgtagaagggaaagtgatttacgaaacccagtccactcacaaactgctggcggcgttctctcaggcttccatgatccacgttaaaggtgacgtaaacgaagaaacctttaacgaagcctacatgatgcacaccaccacttctccgcactacggtatcgtggcgtccactgaaaccgctgcggcgatgatgaaaggcaatgcaggtaagcgtctgatcaacggttctattgaacgtgcgatcaaattccgtaaagagatcaaacgtctgagaacggaatctgatggctggttctttgatgtatggcagccggatcatatcgatacgactgaatgctggccgctgcgttctgacagcacctggcacggcttcaaaaacatcgataacgagcacatgtatcttgacccgatcaaagtcaccctgctgactccggggatggaaaaagacggcaccatgagcgactttggtattccggccagcatcgtggcgaaatacctcgacgaacatggcatcgttgttgagaaaaccggtccgtataacctgctgttcctgttcagcatcggtatcgataagaccaaagcactgagcctgctgcgtgctctgactgactttaaacgtgcgttcgacctgaacctgcgtgtgaaaaacatgctgccgtctctgtatcgtgaagatcctgaattctatgaaaacatgcgtattcaggaactggctcagaatatccacaaactgattgttcaccacaatctgccggatctgatgtatcgcgcatttgaagtgctgccgacgatggtaatgactccgtatgctgcattccagaaagagctgcacggtatgaccgaagaagtttacctcgacgaaatggtaggtcgtattaacgccaatatgatccttccgtacccgccgggagttcctctggtaatgccgggtgaaatgatcaccgaagaaagccgtccggttctggagttcctgcagatgctgtgtgaaatcggcgctcactatccgggctttgaaaccgatattcacggtgcataccgtcaggctgatggccgctataccgttaaggtattgaaagaagaaagcaaaaaataa

SEQ ID NO: 2 - protein, 715 aa, Escherichia coli
MNVIAILNHMGVYFKEEPIRELHRALERLNFQIVYPNDRDDLLKLIENNARLCGVIFDWDKYNLELCEEISKMNENLPLYAFANTYSTLDVSLNDLRLQISFFEYALGAAEDIANKIKQTTDEYINTILPPLTKALFKYVREGKYTFCTPGHMGGTAFQKSPVGSLFYDFFGPNTMKSDISISVSELGSLLDHSGPHKEAEQYIARVFNADRSYMVTNGTSTANKIVGMYSAPAGSTILIDRNCHKSLTHLMMMSDVTPIYFRPTRNAYGILGGIPQSEFQHATIAKRVKETPNATWPVHAVITNSTYDGLLYNTDFIKKTLDVKSIHFDSAWVPYTNFSPIYEGKCGMSGGRVEGKVIYETQSTHKLLAAFSQASMIHVKGDVNEETFNEAYMMHTTTSPHYGIVASTETAAAMMKGNAGKRLINGSIERAIKFRKEIKRLRTESDGWFFDVWQPDHIDTTECWPLRSDSTWHGFKNIDNEHMYLDPIKVTLLTPGMEKDGTMSDFGIPASIVAKYLDEHGIVVEKTGPYNLLFLFSIGIDKTKALSLLRALTDFKRAFDLNLRVKNMLPSLYREDPEFYENMRIQELAQNIHKLIVHHNLPDLMYRAFEVLPTMVMTPYAAFQKELHGMTEEVYLDEMVGRINANMILPYPPGVPLVMPGEMITEESRPVLEFLQMLCEIGAHYPGFETDIHGAYRQADGRYTVKVLKEESKK

SEQ ID NO: 3 - DNA, 1335 bp, Escherichia coli
atgagttctgccaagaagatcgggctatttgcctgtaccggtgttgttgccggtaatatgatggggagcggtattgcattattacctgcgaacctagcaagtatcggtggtattgctatctggggttggattatctctattattggtgcaatgtcgctggcgtatgtatatgcccgactggcaacaaaaaacccgcaacaaggtggcccaattgcttatgccggagaaatttcccctgcatttggttttcagacaggtgttctttattaccatgctaactggattggtaacctggcgattggtattaccgctgtatcttatctttccaccttcttcccagtattaaatgatcctgttccggcgggtatcgcctgtattgctatcgtctgggtatttacctttgtaaatatgctcggcggtacttgggtaagccgtttaaccactattggtctggtgctggttcttattcctgtggtgatgactgctattgttggctggcattggtttgatgcggcaacttatgcagctaactggaatactgcggataccactgatggtcatgcgatcattaaaagtattctgctctgcctgtgggccttcgtgggtgttgaatccgcagctgtaagtactggtatggttaaaaacccgaaacgtaccgttccgctggcaaccatgctgggtactggtttagcaggtattgtttacatcgctgcgactcaggtgctttccggtatgtatccgtcttctgtaatggcggcttccggtgctccgtttgcaatcagtgcttcaactatcctcggtaactgggctgcgccgctggtttctgcattcaccgcctttgcgtgcctgacttctctgggctcctggatgatgttggtaggccaggcaggtgtacgtgccgctaacgacggtaacttcccgaaagtttatggtgaagtcgacagcaacggtattccgaaaaaaggtctgctgctggctgcagtgaaaatgactgccctgatgatccttatcactctgatgaactctgccggtggtaaagcatctgacctgttcggtgaactgaccggtatcgcagtactgctgactatgctgccgtatttctactcttgcgttgacctgattcgttttgaaggcgttaacatccgcaactttgtcagcctgatctgctctgtactgggttgcgtgttctgcttcatcgcgctgatgggcgcaagctccttcgagctggcaggtaccttcatcgtcagcctgattatcctgatgttctacgctcgcaaaatgcacgagcgccagagccactcaatggataaccacaccgcgtctaacgcacattaa

SEQ ID NO: 4 - protein, 444 aa, Escherichia coli
MSSAKKIGLFACTGVVAGNMMGSGIALLPANLASIGGIAIWGWIISIIGAMSLAYVYARLATKNPQQGGPIAYAGEISPAFGFQTGVLYYHANWIGNLAIGITAVSYLSTFFPVLNDPVPAGIACIAIVWVFTFVNMLGGTWVSRLTTIGLVLVLIPVVMTAIVGWHWFDAATYAANWNTADTTDGHAIIKSILLCLWAFVGVESAAVSTGMVKNPKRTVPLATMLGTGLAGIVYIAATQVLSGMYPSSVMAASGAPFAISASTILGNWAAPLVSAFTAFACLTSLGSWMMLVGQAGVRAANDGNFPKVYGEVDSNGIPKKGLLLAAVKMTALMILITLMNSAGGKASDLFGELTGIAVLLTMLPYFYSCVDLIRFEGVNIRNFVSLICSVLGCVFCFIALMGASSFELAGTFIVSLIILMFYARKMHERQSHSMDNHTASNAH

SEQ ID NO: 5 - DNA, 47 bp, Escherichia coli
ttgtcgacaaggagatatagatatgaacgttattgcaatattgaatc

SEQ ID NO: 6 - DNA, 36 bp, Escherichia coli
aaggatccttattttttgctttcttctttcaatacc

SEQ ID NO: 7 - DNA, 44 bp, Escherichia coli
ttggatccaaggagatatagatatgagttctgccaagaagatcg

SEQ ID NO: 8 - DNA, 36 bp, Escherichia coli; identical to SEQ ID NO: 6